(19) United States
(12) Patent Application Publication
(10) Pub. No.: US 2010/0047859 A1
(43) Pub. Date: Feb. 25, 2010

(54) SILICA-BASED FLUORESCENT NANOPARTICLES

(76) Inventor: Jin-Kyu Lee, Seoul (KR)

Correspondence Address: EDWARDS ANGELL PALMER & DODGE LLP, P.O. BOX 55874, BOSTON, MA, 02205, US

(21) Appl. No.: 12/196,808

(22) Filed: Aug. 22, 2008

(51) Int. Cl. G01N 1/30; C08L 83/00; C07D 311/80; C07D 311/78; C07D 311/04; G01N 21/75

(52) U.S. Cl. 435/40.5; 524/588; 549/390; 549/384; 549/399; 436/172

(57) ABSTRACT

A composition including the reaction product of: an organic silane of Formula SiR¹ₘX¹₄₋ₘ; a fluorescent dye-silane compound of Formula D-L′-(CH₂)ₙ—SiX²₃; water; and a hydrolysis catalyst; where R¹ is a C₁-C₆ alkyl that is unsubstituted or substituted with one or more halogens or hydroxyl group, C₂-C₆ alkenyl that is unsubstituted or substituted with one or more halogens or hydroxyl group, or an aryl group that is unsubstituted or substituted with one or more halogens or hydroxyl group, m is 0 or 1; n is 3, 4, 5, 6, 7, 8, 9, 10, 11, or 12, D is a radical having a fluorophore; L1 is a bond, O, S, C(O)O, C(O)NR², SO₂O, C(O)S, C(S), or S₂; R² is hydrogen, a C₁-C₁₂ alkyl that is unsubstituted or is substituted with hydroxyl; each X¹ and X² are independently a hydrolyzable substituent; and the reaction product is a silica-based fluorescent nanoparticle.

### BACKGROUND

Fluorescence results when the molecular absorption of a photon triggers the emission of another photon with a longer wavelength. Fluorescence can also occur when a fluorophore relaxes to its ground state after being electrically excited. Fluorophores are components of a molecule which cause a molecule to be fluorescent. Fluorophores absorb energy of a specific wavelength (or range of wavelengths) and re-emit that energy at a different (but equally specific) wavelength (or range of wavelengths).

Flourescent materials include inorganic materials (e.g. zinc sulfide, among others) and organic materials (e.g. rhodamine, fluorescein, and eosin, among others). Flourescent materials may be included in, for example, fluorescent dyes or pigments, such as those used for fluorescent inks (e.g. in writing instruments, printers, etc.) or fluorescent paints (e.g. indicators, fibers, etc.).

### SUMMARY

In one aspect, a composition is provided including the reaction product of: an organic silane of Formula SiR¹ₘX¹₄₋ₘ; a fluorescent dye-silane compound of Formula D-L¹-(CH₂)ₙ—SiX²₃; water; and a hydrolysis catalyst; where R¹ is a C₁-C₆ alkyl that is unsubstituted or substituted with one or more halogens or hydroxyl group, C₂-C₆ alkenyl that is unsubstituted or substituted with one or more halogens or hydroxyl group, or an aryl group that is unsubstituted or substituted with one or more halogens or hydroxyl group, m is 0 or 1; n is 3, 4, 5, 6, 7, 8, 9, 10, 11, or 12, D is a radical having afluorophore; L1 is a bond, O, S, C(O)O, C(O)NR², SO₂O, C(O)S, C(S), or S₂; R² is hydrogen, a C₁-C₁₂ alkyl that is unsubstituted or is substituted with hydroxyl; each X¹ and X² are independently a hydrolyzable substituent; and the reaction product is a silica-based fluorescent nanoparticle including an outer surface including functional groups. In some embodiments, 80% or more of the functional groups on the outer surface of the silica-based fluorescent nanoparticle are OH groups. In other embodiments, 10% or less of the functional groups on the outer surface of the silica-based fluorescent nanoparticle are NH₂ groups.

In some embodiments, D is a radical having a fluorophore derived from fluorescent dyes based on xanthene, benzo[a]xanthene, benzo[b]xanthene, benzo[c]xanthene, coumarin, benzocoumarin, alizarin, azo, phenoxazine, benzo[a]phenoxazine, benzo[b]phenoxazine, benzo[c]phenoxazine, naphthalimide, naphtholactam, azlactone, methyne, oxazine, thiazine, diketopyrrolopyrrole, quinacridone, thioepindoline, lactamimide, diphenylmaleimide, acetoacetamide, imidazothiazine, benzanthrone, phthalimide, benzotriazole, pyrimidine, pyrazine, and triazine.

In some embodiments, a surface of the silica-based fluorescent includes, a group that is —OR³ or —(CH₂)ₚY bonded to a silicon atom; where Y is NR⁴R⁵R⁶, PO₂(OR⁷), or —(OCH₂CH₂)₄₋₁₅OR⁸, where R³, R⁴, R⁵, R⁶, R⁷, and R⁸ are each independently C₁-C₈ alkyl; and p is an integer from 1 to 15. In some embodiments, R¹ is methyl, ethyl, or phenyl. In other embodiments, X¹ and X² are each independently halogen, C₁-C₆ alkoxy, or C₁-C₆ acyloxy.

In some embodiments, D is a radical represented by Formula I, Formula II, Formula III, or Formula IV:

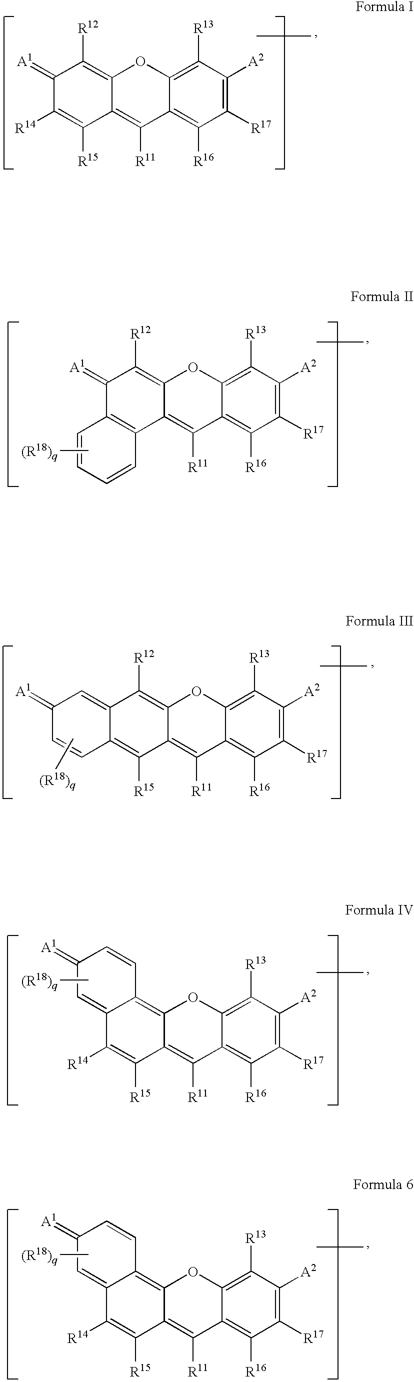

where A¹ is O, N-Z¹, or NZ¹Z²; Z¹ and Z² are each independently H or C₁-C₈ alkyl, or Z¹ and R¹² join together to form a 5-, 6-, or 7-membered ring together with the atoms to which they are bonded, or Z¹ and R¹⁴ join together to form a 5-, 6-, or 7-membered ring together with the atoms to which they are bonded, or Z² and R¹⁴ join together to form a 5-, 6-, or 7-membered ring together with the atoms to which they are bonded; A² is OZ³ or NZ⁴Z⁵; Z³ is H, C₁-C₈ alkyl, or carboxy C₁-C₈ alkyl; Z⁴ and Z⁵ are each independently H or C₁-C₈ alkyl, or Z⁴ and R¹³ join together to form a 5-, 6-, or 7-membered ring together with the atoms to which they are bonded, or Z⁴ and R¹⁷ join together to form a 5-, 6-, or 7-membered ring together with the atoms to which they are bonded, or Z⁵ and R¹⁷ join together to form a 5-, 6-, or 7-membered ring together with the atoms to which they are bonded; q is an integer of 1 to 4, R¹¹ is F, Cl, Br, I, CN, CF₃, C₁-C₈ alkyl, C₂-C₈ alkenyl, C₂-C₈ alkynyl, phenyl, naphthyl, or a group of Formula

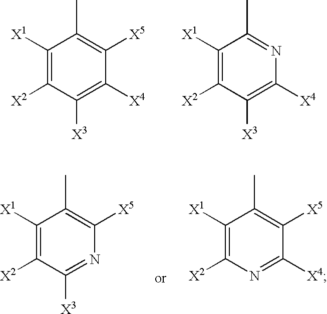

X¹, X², X³, X⁴, and X⁵ are independently H, F, Cl, Br, I, CN, CF₃, C₁-C₈ alkyl, C₁-C₈ alkoxy, C₁-C₈ alkylthio, C₂-C₈ alkenyl, C₂-C₈ alkynyl, C₁-C₈ alkylamido, SO₃H, sulfonate, and CO₂H, or X¹ and X², X² and X³, X³ and X⁴, or X⁴ and X⁵ join together form a phenyl group, together with the atoms to which they are bonded, which is unsubstituted or substituted with 1 to 4 F, Cl, Br, I, CN, CO₂H, SO₃H, OH, NH₂, with unsubstituted or substituted mono- or di(C₁-C₈ alkyl)amino, unsubstituted or substituted C₁-C₈ alkyl, unsubstituted or substituted C₁-C₈ alkylthio, or unsubstituted or substituted C₁-C₈ alkoxy; and R¹², R¹³, R¹⁴, R¹⁵, R¹⁶, R¹⁷, and R¹⁸ are independently H, F, Cl, Br, I, CN, CF₃, unsubstituted or substituted C₁-C₈ alkyl, unsubstituted or substituted C₁-C₈ alkylthio, unsubstituted or substituted C₁-C₈ alkoxy, phenyl, naphthyl, or heteroaryl, or R¹⁴ and R¹⁵, or R¹⁸ and R¹⁷ join to form a benzo group.

In some embodiments, D is a radical represented by Formula V:

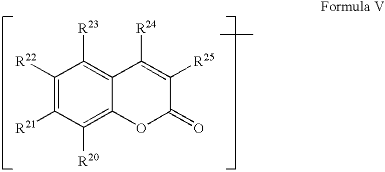

where R²⁰, R²¹, R²², R²³, R²⁴, and R²⁵ are independently H, C₁-C₈ alkyl, and NZ⁶Z⁷, Z⁶ and Z⁷ are each independently H or C₁-C₈ alkyl, or R²⁰ and Z⁶; R²⁰ and Z⁷; R²² and Z⁶; or R²² and Z⁷ join together to form a 5-, 6-, or 7-membered ring together with atoms to which they are bonded, that may be unsubstituted or substituted.

In some embodiments, a ratio of the organic silane to the fluorescent dye-silane ranges from 1:1 to 100:1.

In another aspect, a method of preparing a silica-based florescent nanoparticle is provided including reacting a fluorescent dye of Formula D-A with a compound of Formula B—(CH₂)_{q}—CH═CH₂ to produce a fluorescent dye derivative of Formula D-L1-(CH₂)_{q}—CH═CH₂; reacting the fluorescent dye derivative of Formula D-L1-(CH₂)_{q}—CH═CH₂ with a silane compound of Formula HSiX²₃ to produce a fluorescent dye-silane compound of Formula D-L¹-(CH₂)_{n′}—SiX²₃: and polymerizing an organic silane of Formula SiR¹_{m′}X¹_{4-m′} and the fluorescent dye-silane compound of Formula D-L′-(CH₂)_{n′}—SiX²₃ in the presence of water and a hydrolysis catalyst: where R¹ is a C₁-C₆ alkyl that is unsubstituted or substituted with one or more halogens or hydroxyl group, C₂-C₆ alkenyl that is unsubstituted or substituted with one or more halogens or hydroxyl group, or an aryl group that is unsubstituted or substituted with one or more halogens or hydroxyl group; D is a radical having a fluorophore; each X¹ and X² are independently a hydrolyzable substituent; L1 is a bond, O, S, C(O)O, C(O)NR², SO₂O, C(O)S, C(S), or S₂; A is COOH, OH, SO3H, CO—CH₂-halogen, CH═CH₂ or SH, and B is OH, NHR², F, Cl, Br, I, or SH; q′ is an integer of 1 to 10; n′ is an integer of 3 to 12; m′ is 0 or 1; R² is hydrogen, C₁-C₁₂ alkyl, or hydroxy-substituted C₁-C₁₂ alkyl; with the proviso that A and B are selected in such a manner as to be able to react with each other.

In some embodiments, L′ is C(O)O, A is C(O)OH, and B is F, Cl, Br, I, or OH. In other embodiments, the fluorescent dye derivative of Formula D-L1-(CH₂)_{q}—CH═CH₂ is reacted with the silane compound of Formula HSiX²₃ in at ratio of from 1:0.5 to 1:5.

In another aspect, a compound represented by Formula D-L1-(CH₂)q′-CH═CH₂ is provided where D is a radical having a fluorophore; L1 is a bond, O, S, C(O)O, C(O)NR², SO₂O, C(O)S, C(S), or S₂; and q′ is an integer of 1 to 10. In some embodiments, D is a radical represented by Formula I, Formula II, Formula III, or Formula IV. In some embodiments, D is a radical represented by Formula V:

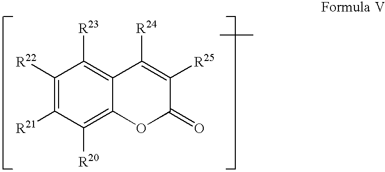

where R²⁰, R²¹, R²², R²³, R²⁴, and R²⁵ are independently H, C₁-C₈ alkyl, and NZ⁶Z⁷; Z⁶ and Z⁷ are each independently H or C₁-C₈ alkyl, or R²⁰ and Z⁶; R²⁰ and Z⁷; R²² and Z⁶; or R²² and Z⁷ join together to form a 5-, 6-, or 7-membered ring together with atoms to which they are bonded, that may be unsubstituted or substituted.

In another aspect, a colorant composition is provided including a silica-based fluorescent nanoparticle. In some embodiments, the colorant composition includes a fluorescent ink, a fluorescent paint or a fluorescent paste. In other embodiments, the colorant composition is used as a biochemical marker.

In another aspect, a method is provided of coloring an article by a silica-based fluorescent nanoparticle.

In another aspect, a method of biochemically staining a cell by using a silica-based fluorescent nanoparticle, is provided.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTIONS OF THE DRAWINGS

FIG. 1 is a photomicrograph (optical microscope magnification: ×200) of an illustrative embodiment of MC3T3-E1 cells labelled with the silica-based fluorescent nanoparticles of Example 1.

FIG. 2 is a photomicrograph (fluorescence microscope magnification: ×200) of an illustrative embodiment of MC3T3-E1 cells labelled with the silica-based fluorescent nanoparticles of Example 1.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Fluorescence has been shown to be an excellent tool for many systems down to the molecular scale, at least in part due to its high signal-to-noise ratio, excellent spatial resolution, and ease of implementation. Described herein, are silica-based fluorescent nanoparticles, synthetic intermediates thereof, surface-modified silica-based fluorescent nanoparticles, and methods of making and using such nanoparticles. The silica-based fluorescent nanoparticles described herein are capable of mass production, and uses include, but are not limited to, article coloring or biochemical markers, for example.

### Silica-Based Fluorescent Nanoparticles

In one aspect, a silica-based fluorescent nanoparticle is provided including the reaction product of an organic silane of Formula SiR¹ₘX¹₄₋ₘ, where R¹ is a C₁-C₆ alkyl, C₂-C₆ alkenyl, or phenyl group that can be substituted with halogen or hydroxy, each X¹ is independently a hydrolyzable substituent, and m is an integer of 0 or 1; and a fluorescent dye-silane compound of Formula D-L¹-(CH₂)ₙ—SiX²₃, where D is a radical having a fluorophore; L¹ is selected from a direct bond, —CO—O—, —CO—NR²—, —O—, —SO₂—O—, —CO—S—, —S—, —CS—, and —S—S—, where R² is hydrogen, C₁-C₁₂ alkyl, or hydroxyl-substituted C₁-C₁₂ alkyl, each X² is independently a hydrolyzable substituent, and n is an integer of 3 to 12, in the presence of water and a hydrolysis catalyst. In some embodiments, the fluorophore that is D is derived from a fluorescent dye such as xanthene-, benzo[a]xanthene-, benzo[b]xanthene-, benzo[c]xanthene-, coumarin-, benzocoumarin-, alizarin-, azo-, phenoxazine-, benzo[a]phenoxazine-, benzo[b]phenoxazine-, benzo[c]phenoxazine-, naphthalimide-, naphtholactam-, azlactone-, methyne-, oxazine-, thiazine-, diketopyrrolopyrrole-, quinacridone-, thioepindoline-, lactamimide-, diphenylmaleimide-, acetoacetamide-, imidazothiazine-, benzanthrone-, phthalimide-, benzotriazole-, pyrimidine-, pyrazine-, and triazine-based fluorescent dyes

In some embodiments, at least about 80%, 85% or 90% of the functional groups on the surface of the silica-based fluorescent nanoparticle are —OH groups. Also, among all groups existing on the surface of the silica-based fluorescent nanoparticle, —NH₂ groups account for 10% or less, 15% or less, or 20% or less. Since —OH groups are 80% or more and/or —NH₂ groups are 20% or less among all groups existing on the surface of the silica-based fluorescent nanoparticles, the nanoparticle is capable of complexing with metal ions, such as cobalt, nickel, copper, cadmium, and mercury ions. In some embodiments, the metal ions are bi-valent.

The silica-based fluorescent nanoparticles have a diameter of about 5 to 900 nanometers, for example, about 30 to 500 nanometers, or 50 to 300 nanometers when measured by means of a transmission electron microscope (TEM) or scanning electron microscope (SEM).

The silica-based fluorescent nanoparticle includes the reaction product obtained by reacting the organic silane of Formula SiR¹ₘX¹₄₋ₘ and the fluorescent dye-silane compound of Formula D-L¹-(CH₂)ₙ—SiX²₃, in the presence of water and a hydrolysis catalyst. The ratio of the organic silane of Formula SiR¹ₘX¹₄₋ₘ to the fluorescent dye-silane compound of Formula D-L¹-(CH₂)ₙ—SiX²₃, which are used as polymerization monomers, is about 1:1 to 100:1, for example 1:1 to 1:50 or 1:1 to 20, according to the various embodiments.

In the organic silane of Formula SiR¹ₘX¹₄₋ₘ, examples of R¹ may include, but are not limited to, methyl, ethyl, and phenyl. In some embodiments, X¹, is a hydrolyzable substituent. Examples of X¹ include, but are not limited to, halogen, C₁-C₆ alkoxy, and C₁-C₆ acyloxy. For example, X¹ may be chloro, methoxy, or ethoxy.

Examples of the organic silane of Formula SiR¹ₘX₄₋ₘ include, but are not limited to, tetramethoxysilane, tetraethoxysilane, chlorotrimethoxysilane, chlorotriethoxysilane, tetrachlorosilane, methyltrimethoxysilane, and methyltriethoxysilane.

In the fluorescent dye-silane compound of Formula D-L¹-(CH₂)ₙ—SiX²₃, D is a radical derived from a fluorescent dye. As used herein, the term “a radical derived from a fluorescent dye” refers to a radical that maintains the parent structure of a fluorescent dye and includes a chromophore essentially imparting fluorescence to the fluorescent dye, i.e., a fluorophore. In some embodiments, L¹ is a direct bond, —CO—O—, —CO—NR²—, or —CS—O—.

In other embodiments, X² is a hydrolyzable substituent may be any substituent that can be hydrolyzed from a silicon atom in the presence of water. Examples of X² include, but are not limited to, halogen, C₁-C₆ alkoxy, and C₁-C₆ acyloxy. For example, X¹ may be chloro, methoxy, or ethoxy. n may be an integer of 3 to 6.

The fluorescent dye may be a xanthene-, benzo[a]xanthene-, benzo[b]xanthene-, benzo[c]xanthene-, coumarin-, benzocoumarin-, alizarin-, azo-, phenoxazine-, benzo[a]phenoxazine-, benzo[b]phenoxazine-, benzo[c]phenoxazine-, naphthalimide-, naphtholactam-, azlactone-, methyne-, oxazine-, thiazine-, diketopyrrolopyrrole-, quinacridone-, thioepindoline-, lactamimide-, diphenylmaleimide-, acetoacetamide-, imidazothiazine-, benzanthrone-, phthalimide-, benzotriazole-, pyrimidine-, pyrazine-, or a triazine-based fluorescent dye.

Examples of D include radicals represented by Formulas I, II, III, and IV:

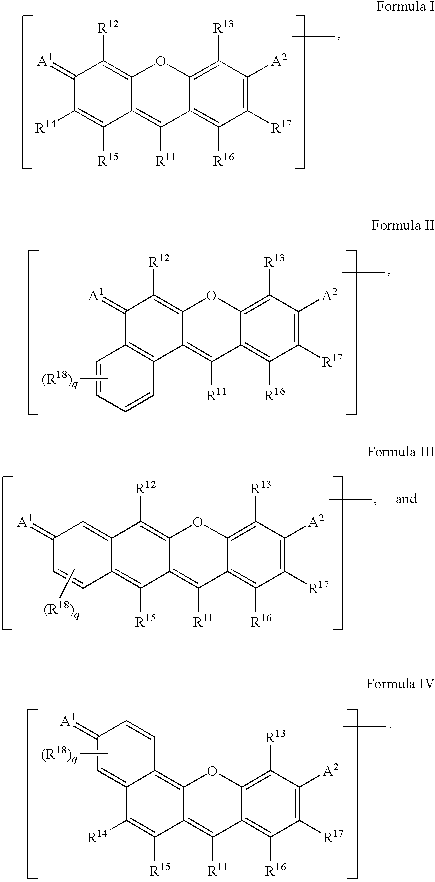

In Formulas I, II, III, and IV, A¹ is O, N-Z¹, or NZ¹Z², where Z¹ and Z² are each independently H or C₁-C₈ alkyl, or alternatively at least one pair selected from R¹² and Z¹; R¹² and Z²; R¹⁴ and Z¹; and R¹⁴ and Z² forms a 5-, 6-, or 7-membered ring together with atoms to which they are bonded. A² is —OZ³ or —NZ⁴Z⁵, where Z³ is H, C₁-C₈ alkyl, or carboxy C₁-C₈ alkyl, and Z⁴ and Z⁵ are each independently H or C₁-C₈ alkyl, or alternatively at least one pair selected from R¹³ and Z⁴; R¹³ and Z⁵; R¹⁷ and Z⁴; and R¹⁷ and Z⁵ forms a 5-, 6-, or 7-membered ring together with atoms to which they are bonded. Variable “q” is 1, 2, 3, or 4. R¹¹ is halogen, cyano, CF₃, C₁-C₈ alkyl, C₂-C₈ alkenyl, C₂-C₈ alkynyl, phenyl, naphthyl, or a group of Formula

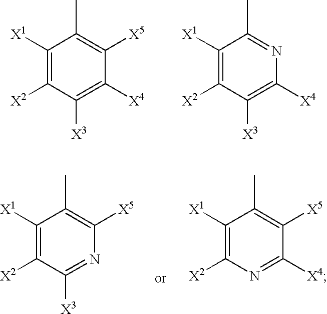

where X¹, X², X³, X⁴, and X⁵ are each independently selected from H, halogen, cyano, CF₃, C₁-C₈ alkyl, C₁-C₈ alkoxy, C₁-C₈ alkylthio, C₂-C₈ alkenyl, C₂-C₈ alkynyl, C₁-C₈ alkylamido, SO₃H, sulfonate, CO₂H, or alternatively two adjacent X¹ to X⁵ substituents together form fused phenyl group which may be substituted with 1 to 4 substituents selected from halogen, cyano, carboxy, sulfo, hydroxy, amino, mono- or di(C₁-C₈ alkyl)amino, C₁-C₈ alkyl, C₁-C₈ alkylthio, and C₁-C₈ alkoxy, where the alkyl part of X¹ to X⁵ may be substituted with halogen, carboxy, sulfo, amino, mono- or di(C₁-C₈ alkyl)amino, C₁-C₈ alkoxy, cyano, haloacetyl, or hydroxy. R¹², R¹³, R¹⁴, R¹⁵, R¹⁶, R¹⁷, and R¹⁸ are each independently selected from H, halogen, cyano, CF₃, C₁-C₈ alkyl, C₁-C₈ alkylthio, C₁-C₈ alkoxy, phenyl, naphthyl, and heteroaryl, where the alkyl part of R¹² to R¹⁸ may be substituted with halogen, carboxy, sulfo, sulfonate, amino, mono- or di(C₁-C₈ alkyl)amino, C₁-C₄ alkoxy, cyano, haloactyl, or hydroxy, and the phenyl, naphthyl, or heteroaryl part of R¹² to R¹⁸ may be substituted with 1 to 4 substituents selected from halogen, cyano, carboxy, sulfo, hydroxy, amino, mono- or di(C₁-C₈)alkylamino, C₁-C₈ alkyl, C₁-C₈ alkylthio, and C₁-C₈ alkoxy. Alternatively, at least one pair of R¹⁴ and R¹⁵; and R¹⁶ and R¹⁷ forms a benzo group together with atoms to which they are bonded.

According to some embodiments, examples of a xanthene-based radical D and a benzoxanthene-based radical D include, but are not limited to, a radical derived from a rhodamine-based fluorescent dye, such as rhodamine B, tetramethylrhodamine (TMR), carboxytetramethylrhodamine (TAMRA), sulforhodamine, sulforhodamine 101 sulfonyl chloride (Texas Red), carboxy-X-rhodamine (ROX), diaminorhodamine, N-(2-aminoethyl)rhodamine 6G-amide bis(trifluoroacetate), N-[2-(2-aminoethylamino)ethyl]rhodamine 6G-amide bis(trifluoroacetate), and N-[4-(aminomethyl)benzyl]rhodamine 6G-amide bis(trifluoroacetate); and a fluorescein-based fluorescent dye, such as fluorescein, aminophenylfluorescein, hydroxyphenylfluorescein, 6-[fluorescein-5(6)-carboxamido]hexanoic acid, (iodoacetamido)fluorescein, 5-(bromomethyl)fluorescein, 1-(O′-methylfluoresceinyl)piperidine-4-carboxylic acid, fluorescein-O′-acetic acid, O′-(carboxymethyl)fluoresceinamide, 5-(4,6-dichloro-s-triazine-2-ylamino)fluorescein, and eosin Y.

An example of where D is a coumarin based-radical is represented by Formula V:

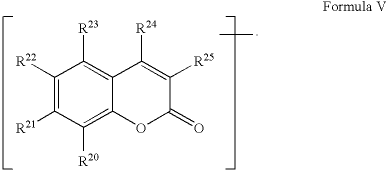

In Formula V, R²⁰, R²¹, R²², R²³, R²⁴, and R²⁵ are each independently H, C₁-C₈ alkyl, and NZ⁶Z⁷, where Z⁶ and Z⁷ are each independently H or C₁-C₈ alkyl, or alternatively at least one pair selected from R²⁰ and Z⁶; R²⁰ and Z⁷; R²² and Z⁶; and R²² and Z⁷ forms a 5-, 6-, or 7-membered ring together with atoms to which they are bonded.

Examples of a coumarin-based radical D include, but not limited to, radicals derived from 7-amino-4-methylcoumarin, coumarin-3-carboxylic acid, coumarin 343, coumarin-6-sulfonyl chloride, 3-(bromoacetyl)coumarin, 7-(diethylamino)coumarin-3-carbohydrazide, and 7-(diethylamino)coumarin-3-carboxilic acid.

Examples of an alizarin-based radical D include, but are not limited to, radicals derived from alizarin (Mordant Red), alizarine-3-methyliminodiacetic acid, 4-[[4-hydroxy-9,10-dioxo-3-[(4-sulfonatophenyl)amino]anthracene-1-yl]amino]benzensulfonic acid disodium salt (Alizarin Blue Black B), and 3,4-dihydroxy-9,10-dioxo-2-anthracenesulfonic acid sodium salt (Alizarin Red S).

Examples of an azo-based radical D include, but are not limited to, radicals derived from 5-(3-nitrophenylazo)salicylic acid sodium salt (Alizarin Yellow GG), 5-(4-nitrophenylazo)salicylic acid (Mordant Orange 1), and 4-hydroxy-3-[(2-hydroxy-1-naphthalenyl)azo]-benzenesulfonic acid sodium salt (Acid Alizarin Violet N).

In addition to the organic silane and the fluorescent dye-silane compound, water is involved in the polymerization. Water may be used in an excess amount sufficient to hydrolyze X¹ and X², the hydrolyzable substituents bonded to silicon atoms of the organic silane and the fluorescent dye-silane compound.

The hydrolysis catalyst may be any organic or inorganic acid or base known in the art to catalyze the hydrolysis of substituents from silicon atoms in the presence of water. The hydrolysis catalyst may be an inorganic base such as, but not limited to, sodium hydroxide, potassium hydroxide, or an aqueous ammonia solution. The hydrolysis catalyst may be an organic acid such as, but not limited to, hydrochloric acid, sulfuric acid, or nitric acid. In some embodiments, the hydrolysis catalyst is aqueous ammonia solution or hydrochloric acid. The hydrolysis catalyst may be separately added to the reaction mixture, or in the case where X¹ of the organic silane is halogen the catalyst may be at least partially generated in situ. The amount of the hydrolysis catalyst used will depend on the chemical composition of the catalyst as well as the temperature at which the hydrolysis reaction occurs, and can be any amount required to effect the hydrolysis completely. In general, the amount of the hydrolysis catalyst may be about 0.02 to 0.5 mole equivalents based upon the molar amounts of the organic silane and/or the fluorescent dye-silane compound. For example, the amount of the hydrolysis catalyst may be within a range of about 0.1 to 0.3 mole equivalent.

### Preparation and Intermediate of Silica-Based Fluorescent Nanoparticle

One aspect provides methods for making the silica-based fluorescent nanoparticle described herein. The silica-based fluorescent nanoparticle may be prepared by polymerizing the organic silane of Formula SiR¹ₘX¹₄₋ₘ and the fluorescent dye-silane compound of Formula D-L¹-(CH₂)ₙ—SiX²₃, in the presence of water and a hydrolysis catalyst. In such Formulas R¹, X¹, X², D, L¹, m, and n are the same as defined above. The equivalent ratio of the organic silane of Formula SiR¹ₘX¹₄₋ₘ to the fluorescent dye-silane compound of Formula D-L¹-(CH₂)ₙ—SiX²₃, which are used as polymerization monomers, may range from about 1:1 to 100:1.

The fluorescent dye-silane compound of Formula D-L¹-(CH₂)ₙ—SiX²₃ may be prepared by reacting a fluorescent dye represented by Formula D-A with a compound represented by Formula B—(CH₂)_{q}—CH═CH₂ to produce a fluorescent dye derivative represented by Formula D-L¹-(CH₂)_{q}—CH═CH₂. In such formulas D and L¹ are the same as defined above, A is —COOH, —OH, —SO₃H, —CO—CH₂-halogen, —CH═CH₂ or —SH, and B is —OH, —NHR², -halogen, or —SH, where R² is hydrogen, C₁-C₁₂ alkyl, or hydroxy-substituted C₁-C₁₂ alkyl, with the proviso that A and B are selected in such a manner as to be able to react with each other, and q′ is an integer of 1 to 10; and reacting the fluorescent dye derivative of Formula D-L¹-(CH₂)_{q}—CH═CH₂ with a silane compound represented by Formula HSiX²₃ to prepare the fluorescent dye-silane compound of Formula D-L¹-(CH₂)ₙ—SiX²₃.

In reacting a fluorescent dye represented by Formula D-A with a compound represented by Formula B—(CH₂)_{q}—CH═CH₂, A and B are selected in such a manner as to be able to react with each other to form another functional group. For example, for a case where L¹ is —CO—O—, —COOH may be selected as A, and -halogen or —OH may be selected as B. As another example, for a case where L¹ is —CO—NR²—, —COOH may be selected as A, and —NHR² may be selected as B. As another example, for a case where L¹ is —O—, —OH may be selected as A, and —OH may be selected as B. As another example, for a case where L¹ is —SO₂—O—, —SO₃OH may be selected as A, and —OH may be selected as B. As another example, for a case where L¹ is —CO—S—, —COOH may be selected as A, and —SH may be selected as B. As another example, for a case where L¹ is —S—, —CO—CH₂-halogen may be selected as A, and —SH may be selected as B. As another example, for a case where L¹ is —CS—, —CH═CH₂ may be selected as A, and —SH may be selected as B. As another example, and for a case where L¹ is —S—S—, —SH may be selected as A, and —SH may be selected as B.

Appropriate reaction conditions for the reaction between functional groups of A and B, including a catalyst, a solvent, and a temperature, are known to those skilled in the art (e.g. see Carey, F. A., Sundberg, R. J., Advanced Organic Chemistry, Part B: Reaction and Synthesis, 5th ed., Springer, 2007; Greg T. Hermanson, Bioconjugate Technique, Academic Press, 1996)).

Reacting the fluorescent dye derivative of Formula D-L¹-(CH₂)_{q}—CH═CH₂ with a silane compound represented by Formula HSiX²₃ includes the hydrosilylation reaction between the fluorescent dye derivative of Formula D-L¹-(CH₂)_{q}—CH═CH₂ and the silane compound of Formula HSiX²₃. This hydrosilylation may be carried out in the presence of a platinum catalyst.

The platinum catalyst may be, but is not limited to, Pt/C, Pt/Al, PtO₂, or chloroplatinic acid. Examples of a solvent for this reaction may include, but are not limited to, alcohol (such as, but limited to, methanol, ethanol and isopropanol), and toluene. For example, this reaction may be carried out at a temperature of about 60° C. to 100° C. for 24 to 36 hours. The equivalent ratio of the fluorescent dye derivative of Formula D-L¹-(CH₂)_{q}—CH═CH₂ to the silane compound of Formula HSiX²₃ amounts to about 1:0.5 to about 1:5, for example, about 1:0.8 to about 1:2.0.

In some embodiments, the method includes an esterification reaction between a fluorescent dye in the form of carboxylic acid and halide or alcohol. In this esterification reaction, an inorganic base may be used as a catalyst. Examples of the esterification catalyst may include, but are not limited to, potassium carbonate, cesium carbonate, etc. In an illustrative embodiment of the method, L¹ may be —CO—O—, A may be —COOH, and B may be -halogen or —OH.

In another aspect, a compound of Formula D-L¹-(CH₂)_{q}—CH═CH₂ is provided. The compound of Formula D-L¹-(CH₂)_{q}—CH═CH₂ is useful as an intermediate in preparing the silica-based fluorescent nanoparticles, and as such may also be used as a fluorescent dye.

### Surface-Modified Silica-Based Fluorescent Nanoparticle and Preparation Method Thereof

The silica-based fluorescent nanoparticle described herein may be surface-modified by any suitable functional group using one or more surface modification scheme known in the art.

In one aspect, the one or more silica-based fluorescent nanoparticles further include one or more radicals -M such as —OR³ and —(CH₂)ₚ—Y, where M is bonded to a silicon atom on the surface of the silica-based fluorescent nanoparticle. In some embodiments, Y is NR⁴R⁵R⁶, —PO₂(OR⁷), or —(O—CH₂—CH₂)₄₋₁₅—OR⁸, where R³, R⁴, R⁵, R⁶, R⁷, and R⁸ are each independently C₁-C₈ alkyl, and p is an integer of 1 to 15.

In another aspect, when the radical -M is —OR³, the surface-modified silica-based fluorescent nanoparticle optionally functions as a binder in a colorant composition. In such compositions a separate binder is optional. The surface-modified silica-based fluorescent nanoparticle in which the radical -M is —OR³ may be obtained, for example, by reacting one or more silica-based fluorescent nanoparticles with R³OH in the presence of an acid or base catalyst. The amount of R³OH may vary according to the desired degree of surface modification, but about 1 to 10 equivalents or 1 to 5 equivalents of R³OH may be generally used per equivalent of the silica-based fluorescent nanoparticle.

In another aspect when the radical -M is —(CH₂)ₚ—Y, and Y is —NR⁴R⁵R⁶, the surface-modified silica-based fluorescent nanoparticles may function as a cationic surface for gene transfer(see Dhruba J. Bharali, et. Al., P. Natl. Acad. Sci. USA 2005, 102, 11539-11544.; W. Tan, et. al., Med. Res. Rev. 2004, 24, 621-638.; T.-J. Yoon, et. al., Angew. Chem. Int. Ed. 2005, 44, 1068-1071.; V. Sokolova and M. Epple, Angew. Chem. Int Ed. 2008, 47, 1382-1395). The transferred gene may be an anionic gene. The surface-modified silica-based fluorescent nanoparticle in which the radical -M is —(CH₂)ₚ—NR⁴R⁵R⁶ may be obtained, for example, by reacting the silica-based fluorescent nanoparticles with [X¹₃Si—(CH₂)ₚ—NR⁴R⁵R⁶]⁺G⁻ in the presence of water and a hydrolysis catalyst. X¹ is a hydrolyzable group as defined above, and G⁻ is a monovalent anion that may be, for example, F, Br, or Cl. With regard to this, the amount of [X¹₃Si—(CH₂)ₚ—NR⁴R⁵R⁶]⁺G⁻ may vary according to the desired degree of surface modification, but about 1 to 20 equivalents of [X¹₃Si—(CH₂)ₚ—NR⁴R⁵R⁶]⁺G⁻ may be generally used per equivalent of the silica-based fluorescent nanoparticle. For example, [X¹₃Si—(CH₂)ₚ—NR⁴R⁵R⁶]⁺G⁻ may be (CH₃O)₃Si—(CH₂)₃—N⁺(CH₃)₃Cl⁻, that is, (CH₃O)₃Si-PTMA.

In one aspect, when the radical -M is —(CH₂)ₚ—Y, and Y is —PO₂(OR⁷), the surface-modified silica-based fluorescent nanoparticles may function as an anionic surface for a comparison of PEG and a cation(see Dhruba J. Bharali, et. Al., P. Natl. Acad. Sci. USA 2005, 102, 11539-11544.; W. Tan, et. al., Med. Res. Rev. 2004, 24, 621-638.; T.-J. Yoon, et. al., Angew. Chem. Int. Ed. 2005, 44, 1068-1071.; V. Sokolova and M. Epple, Angew. Chem. Int. Ed. 2008, 47, 1382-1395). The surface-modified silica-based fluorescent nanoparticle in which the radical -M is —(CH₂)_{p—PO}₂(OR⁷) may be obtained, by reacting the silica-based fluorescent nanoparticles with Q⁺[X¹₃Si—(CH₂)ₚ—PO₂(OR⁷)]⁻ in the presence of water and a hydrolysis catalyst. In such formulas, X¹ is a hydrolyzable group as defined above, and Q⁺ is a monovalent cation that may be, for example, Na, K, or NH₄. With regard to this, the amount of Q⁺[X¹₃Si—(CH₂)ₚ—PO₂(OR⁷)]⁻ may vary according to the desired degree of surface modification, but about 1 to 20 mol equivalents of Q⁺[X¹₃Si—(CH₂)ₚ—PO₂(OR⁷)]⁻ may be generally used per mol equivalent of the silica-based fluorescent nanoparticle. For example, Q⁺[X¹₃Si—(CH₂)ₚ—PO₂(OR⁷)]⁻ may be (CH₃O)₃Si—(CH₂)₃—PO₂(OCH₃)Na, that is, (CH₃O)₃Si—PMP.

In one aspect when the radical -M is —(CH₂)ₚ—Y, and Y is —(OCH₂CH₂)₄₋₁₅—OR⁸, the surface-modified silica-based fluorescent nanoparticles may function as a biocompatible surface(see Dhruba J. Bharali, et. Al., P. Natl. Acad. Sci. USA 2005, 102, 11539-11544.; W. Tan, et al., Med. Res. Rev. 2004, 24, 621-638.; T.-J. Yoon, et al., Angew. Chem. Int. Ed. 2005, 44, 1068-1071.; V. Sokolova and M. Epple, Angew. Chem. Int. Ed. 2008, 47, 1382-1395). The surface-modified silica-based fluorescent nanoparticle in which the radical -M is —(CH₂)ₚ—(OCH₂CH₂)₄₋₁₅—OR⁸ may be obtained, by reacting the silica-based fluorescent nanoparticles with X¹₃Si—(CH₂)ₚ—(OCH₂CH₂)₄₋₁₅—OR⁸ in the presence of water and a hydrolysis catalyst (X¹ is a hydrolyzable group as defined above). With regard to this, the amount of X¹₃Si—(CH₂)ₚ—(OCH₂CH₂)₄₋₁₅—OR⁸ may vary according to the desired degree of surface modification, but about 1 to 10 mol equivalents of X¹₃Si—(CH₂)ₚ—(OCH₂CH₂)₄₋₁₅—OR⁸ may be generally used per mol equivalent of the silica-based fluorescent nanoparticle. For example, X¹₃Si—(CH₂)ₚ—(OCH₂CH₂)₄₋₁₅—OR⁸ may be (CH₃O)₃Si—(CH₂)₃—(OCH₂CH₂)₄₋₁₅—OCH₃, that is, (CH₃O)₃Si-PEG.

### Use of Silica-Base Fluorescent Nanoparticles

In another aspect, uses of the silica-based fluorescent nanoparticle or the surface-modified silica-based fluorescent nanoparticle, are provided. In some embodiments, compositions including one or more of the silica-based fluorescent nanoparticles and/or one or more of the surface-modified silica-based fluorescent nanoparticles are provided. Such compositions may include, but are not limited to, fluorescent inks, fluorescent paints or fluorescent pastes. For example, fluorescent inks may be used as an ink for a fluorescent writing instrument, and fluorescent paints may be used for producing fluorescent markers, fluorescent fibers or fluorescent decorations.

The silica-based fluorescent nanoparticle or the surface-modified silica-based fluorescent nanoparticles are able to be prepared at low cost without using expensive fluorescent dyes. Further, the fluorescent dye-derived chromophore is embedded in the silica matrix, which although not wishing to be limited to a particular mechanism, can prevent or at least minimize the fluorescence from being deteriorated by attacks from chemical substances, oxidation in air, and so forth, as compared to a colorant composition including a bare fluorescent dye. In addition, although there is no intention to limit the scope of the present disclosure to a particular theory, the fluorescent dye-derived chromophore has much higher photostability than that of a fluorescent dye in a solution due to restricted irradiative motion, (see C. Earhart, N. R. Jana, N. Erathodiyil, J. Y. Ying, Langmuir 2008, 24, 6215 (2008); K. P Mcnamara et al. Anal. Chem. 70, 4853 (1998); S. Santra et al. Anal. Chem. 73, 4988 (2001); T.-J. Yoon et al. Angew. Chem. Int. Ed. 44, 1068 (2005)).

In colorant compositions of the silica-based fluorescent nanoparticles, the nanoparticle or the surface-modified fluorescent nanoparticle may be included in the amount of 1 to 30% by weight, for example, 1 to 20% by weight or 1 to 25% by weight, based on the total weight of the colorant composition.

The silica-based fluorescent nanoparticle or the surface-modified silica-based fluorescent nanoparticle included in the colorant compositions may have a diameter of about 5 to 900 nanometers, for example, about 30 to 500 nanometers or 50 to 300 nanometers, when measured by means of a transmission electron microscope (TEM) or scanning electron microscope (SEM). There is not a limit to the shape of the nanoparticle. For example, the nanoparticle may be approximately spherical or ellipsoidal.

In some embodiments, the colorant compositions include one or more solvents. The solvent may be included in an amount of 60 to 99% by weight, for example, 70 to 99% by weight, or 70 to 80% by weight based on the total weight of the colorant composition. The solvent may be water or an organic solvent or a mixture thereof. Examples of the organic solvent include, but are not limited to, alcohol, alkyl ether of polyhydric alcohol, a heterocyclic ring-containing organic solvent, and a mixture thereof. In some embodiments, the solvent may work with biological systems, as well as being a buffer.

Examples of the alcohol include, but are not limited to, C₁-C₄ alcohols and alkanediols. Examples of C₁-C₄ alcohols include methanol, ethanol, 1-propanol, 2-propanol, 1-butanol, 2-methyl-1-propanol, and tert-butanol. Examples of the alkanediols include 1,2-alkanediols (e.g. 1,2-pentanediol, 1,2-hexanediol), terminal diols (e.g. 1,4-butanediol, 1,5-pentanediol, 1,6-hexanediol, etc.), and branched diols (e.g. 2,2-dimethyl-1,3-propanediol, 2-methyl-1,4-butanediol, 2-methy-2,4-pentanediol, 3-methyl-1,5-pentanediol, 3-methyl-1,3-butanediol, 2-methyl-1,3-propanediol, 2,5-dimethyl-2,5-hexanediol), or a mixture of any two or more.

Examples of alkyl ethers of polyhydric alcohols include lower monoalkyl ethers of polyhydric alcohols (e.g. monomethyl ether, monoethyl ether, mono-n-butyl ether, mono-iso-buytl ether, mono-n-hexyl ether, etc. of ethyleneglycol, diethyleneglycol, triethyleneglycol, tetraethyleneglycol, propyleneglycol, dipropyleneglycol, and tripropyleneglycol), and lower dialkyl ethers of polyhydric alcohols (e.g. dimethyl ether, diethyl ether, di-n-butyl ether, di-iso-butyl ether, di-n-hexyl ether, etc. of ethyleneglycol, diethyleneglycol, triethyleneglycol, propyleneglycol, dipropyleneglycol, and tripropyleneglycol), or a mixture of any two or more.

Examples of the heterocyclic ring-containing organic solvents include 2-pyrrolidone, ε-caprolactam, tetrahydrofuran, 1,4-dioxane, 1,3-dimethylimidazolidinone (e.g. 1,3-dimethylimidazolidine-2-one), N-methylpyrrolidone, ethyleneurea, sulfolane, pyridine, pyrazine, morpholine, 1-methyl-2-pyridone, 2-methyl-2-oxazoline, 2-ethyl-2-oxazoline, and 2,4,4-trimethyl-2-oxazoline, or a mixture of any two or more.

In some embodiments, the organic solvent is diethyleneglycol, triethyleneglycol, glycerin, triethyleneglycol monobutyl ether, 1,5-pentanediol, 1,2-haxanediol, 2-propanol, triethanolamine, 2-pyrrolidone, or a mixture of any two or more.

In addition to the silica-based fluorescent nanoparticle and the solvent, the colorant compositions may further include known additives generally used in a colorant composition, such as a surfactant, a dispersant, a binder, a viscosity modifier, and the like. Each of these additives may be included in the amount of 0.005 to 5% by weight, for example, 0.01 to 2% by weight, based on the total weight of the colorant composition.

The surfactant can, for example, improve the water resistance of a painted image and prevent smearing of the painted image by adjusting the liquidity (e.g. surface tension) of the colorant composition. Examples of the surfactants include an anionic surfactant (e.g. a fatty acid salt, an alkylsulfonic acid ester salt, alkylbenzene sulfonate, alkylnaphthalene sulfonate, dialkylsulfosuccinate, an alkylphosphoric acid ester salt, a naphthalenesulfonic acid-formalin condensate, a polyoxyethylene alkylsulfuric acid ester salt, etc.), a cationic surfactant (e.g. a fatty amine salt, a quaternary ammonium salt, an alkylpyridinium salt, etc.), a non-ionic surfactant (e.g. polyoxyethylene alkyl ether, polyoxyethylene alkyl aryl ether, polyoxyethylene fatty acid ester, sorbitan fatty acid ester, polyoxyethylene sorbitan fatty acid ester, polyoxyethylene alkyl amine, glycerin fatty acid ester, an oxyethylene-oxypropylene block copolymer, an acetylene-based polyoxyethylene oxide, etc.), an amphoteric surfactant, such as amino acid-type and betaine-type surfactants, a fluorine-based compound, and silicon-based compound, or a mixture of any two or more.

Dispersants contribute to the storage stability of the colorant composition by improving the dispersion of the silica-based fluorescent nanoparticle within the colorant composition. Examples of the dispersants may include a water-soluble acrylic resin, a crosslinked water-soluble acrylic resin, a water-soluble maleic resin, a water-soluble styrene resin, a water-soluble styrene acrylic resin, a water-soluble sttrene maleic resin, polyvinylpyrrolidone, polyvinyl alcohol, and a water-soluble urethane resin, or a mixture of any two or more.

Binders can provide the silica-based fluorescent nanoparticle with coherence, and provide a coated surface with water resistance. Examples of the binder include, but not limited to, polyvinyl alcohol, polyvinyl pyrrolidone, a vinylpyrrolidone-vinylacetate copolymer, a polyvinylacetate-based resin, a polyacrylate-based copolymer, artificial latex, a copolymer of polyvinylacetal, polyvinylacetate, and vinylacetate, and a copolymer of ethylene and vinylacetate, or a mixture of any two or more. In addition, the surface-modified silica-based fluorescent nanoparticles, further including the radical —OR³ bonded to a silicon atom on the surface of the nanoparticle, may be as such used as the binder in the colorant composition. Thus, a colorant composition including such surface-modified silica-based fluorescent nanoparticle may not include a separate binder.

Examples of viscosity modifiers include a water-soluble polymer (e.g. celluloses and polyvinyl alcohol) and a non-ionic surfactant, as well as the above-mentioned organic solvents, or a mixture of any two or more.

In another aspect, a method of coloring an article by using the silica-based fluorescent nanoparticles or the surface-modified silica-based fluorescent nanoparticles is provided. Examples of the articles include, but not limited to, paper, natural or artificial resins, metals, alloys, glasses, ceramics, and fibers.

The colorant compositions, including the silica-based fluorescent nanoparticle or the surface-modified silica-based fluorescent nanoparticle, may be used as a biochemical marker. For example, the silica-based fluorescent nanoparticle or the surface-modified silica-based fluorescent nanoparticle may be used as a staining agent for a cell, a gene, a nucleic acid, or an antibody.

In another aspect, a method of biochemically staining a cell by using the silica-based fluorescent nanoparticle or the surface-modified silica-based fluorescent nanoparticle is provided. The method of biochemically staining a cell may include bringing the silica-based fluorescent nanoparticle or the surface-modified silica-based fluorescent nanoparticle into contact with a cell. The method may include conjugating the silica-based fluorescent nanoparticle or the surface-modified silica-based fluorescent nanoparticle to an antibody to a cell to be stained, if necessary, before the silica-based fluorescent nanoparticle or the surface-modified silica-based fluorescent nanoparticle comes into contact with the cell.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### Definitions

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The embodiments, illustratively described herein may suitably be practiced in the absence of any element or elements, limitation or limitations, not specifically disclosed herein. Thus, for example, the terms “including,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed invention. Additionally the phrase “consisting essentially of” will be understood to include those elements specifically recited and those additional elements that do not materially affect the basic and novel characteristics of the claimed invention. The phrase “consisting of” excludes any element not specifically specified.

In general, “substituted” refers to a group, as defined below (e.g., an alkyl or aryl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group will be substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, alkynoxy, aryloxy, aralkyloxy, heterocyclyloxy, and heterocyclylalkoxy groups; carbonyls(oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Alkyl groups include straight chain and branched alkyl groups having from 1 to 20 carbon atoms or, in some embodiments, from 1 to 12, 1 to 8, 1 to 6, or 1 to 4 carbon atoms. Alkyl groups further include cycloalkyl groups. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above. Where the term haloalkyl is used, the alkyl group is substituted with one or more halogen atoms.

Alkenyl groups include straight and branched chain and cycloalkyl groups as defined above, except that at least one double bond exists between two carbon atoms. Thus, alkenyl groups have from 2 to about 20 carbon atoms, and typically from 2 to 12 carbons or, in some embodiments, from 2 to 8, 2 to 6, or 2 to 4 carbon atoms. In some embodiments, alkenyl groups include cycloalkenyl groups having from 4 to 20 carbon atoms, 5 to 20 carbon atoms, 5 to 10 carbon atoms, or even 5, 6, 7, or 8 carbon atoms. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl, among others. Representative substituted alkenyl groups may be mono-substituted or substituted more than once, such as, but not limited to, mono-, di- or tri-substituted with substituents such as those listed above.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include mono-, bicyclic and polycyclic ring systems, such as, for example bridged cycloalkyl groups as described below, and fused rings, such as, but not limited to, decalinyl, and the like. In some embodiments, polycyclic cycloalkyl groups have three rings. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4- 2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. In some embodiments, cycloalkylalkyl groups have from 4 to 20 carbon atoms, 4 to 16 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain and cycloalkyl groups as defined above, except that at least one double bond exists between two carbon atoms. Thus, alkenyl groups have from 2 to about 20 carbon atoms, and typically from 2 to 12 carbons or, in some embodiments, from 2 to 8, 2 to 6, or 2 to 4 carbon atoms. In some embodiments, alkenyl groups include cycloalkenyl groups having from 4 to 20 carbon atoms, 5 to 20 carbon atoms, 5 to 10 carbon atoms, or even 5, 6, 7, or 8 carbon atoms. Examples include, but are not limited to vinyl, allyl, CH═CH(CH₃), CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl, among others. Representative substituted alkenyl groups may be mono-substituted or substituted more than once, such as, but not limited to, mono-, di- or tri-substituted with substituents such as those listed above.

Cycloalkenylalkyl groups are alkyl groups as defined above in which a hydrogen or carbon bond of the alkyl group is replaced with a bond to a cycloalkenyl group as defined above. Substituted cycloalkylalkenyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8, 2 to 6, or 2 to 4 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃), among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups include monocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. In some embodiments, aralkyl groups contain 7 to 20 carbon atoms, 7 to 14 carbon atoms or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-ethyl-indanyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. In some embodiments, heterocyclyl groups include 3 to 20 ring members, whereas other such groups have 3 to 6, 3 to 10, 3 to 12, or 3 to 15 ring members. Heterocyclyl groups encompass unsaturated, partially saturated and saturated ring systems, such as, for example, imidazolyl, imidazolinyl and imidazolidinyl groups. The phrase “heterocyclyl group” includes fused ring species including those including fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl(pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl(azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridyl, pyridazinyl, pyrimidinyl, pyrazinyl, thiophenyl, benzothiophenyl, furanyl, benzofuranyl, indolyl, azaindolyl(pyrrolopyridyl), indazolyl, benzimidazolyl, imidazopyridyl(azabenzimidazolyl), pyrazolopyridyl, triazolopyridyl, benzotriazolyl, benzoxazolyl, benzothiazolyl, benzothiadiazolyl, imidazopyridyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups. Although the phrase “heteroaryl groups” includes fused ring compounds such as indolyl and 2,3-dihydro indolyl, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, 4-ethyl-morpholinyl, 4-propylmorpholinyl, furan-2-yl methyl, furan-3-yl methyl, pyridine-3-yl methyl, tetrahydrofuran-2-yl ethyl, and indol-2-yl propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

As used herein, halogen can refer to F, Cl, Br, or I.

As used herein, ammonium, or quaternary amine, refers to groups or ions having the following structure, ⁺NR^{a}R^{b}R^{c}R^{d}, where R^{a}, R^{b}, R^{c}, and R^{d} are independently selected from H and alkyl groups. Thus, all of the R^{a-d} groups may be the same or different. Alkyl ammonium refers to ammonium groups having one, two, three, or four alkyl groups, while tetralkylammonium refers to ammonium groups having four alkyl groups. Mixed alkyl ammoniums are those ammonium having two, three, or four alkyl groups where at least one of the alkyl groups is different from the other alkyl groups.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present embodiments, thus generally described, will be understood more readily by reference to the following examples, which are provided by way of illustration and are not intended to be limiting of the present technology in any way.

### EXAMPLES

The present technology is further illustrated by the following examples, which should not be construed as limiting in any way.

### Example 1

### Preparation of Rhodamine-Derived Silica-Based Fluorescent Nanoparticles

Synthesis of Allyl-Rhodamine B. Rhodamine B (0.5 g, 1.0 mmol), cesium carbonate (1.5 g, 3.26 mmol), and allyl bromide (0.54 g, 4.46 mmol) were dissolved in N,N-dimethylformamide (20 mL). The mixture was stirred at 60° C. for 24 hours. Subsequently, the reaction mixture was mixed with methylene chloride (100 ml) and water (200 ml). The methylene chloride layer was separated, and the solvent evaporated using a rotary evaporator. The resultant product was separated using a 30×5 cm, silica gel 60 column with 1:1 methylene chloride:methanol to obtain 0.511 g of allyl-rhodamine B as a reddish black solid (yield: 94.6%).

¹H NMR (300 MHz, Bruker, CDCl₃); δ ppm 8.31, 7.82, 7.34, 7.10, 6.90, 5.70, 5.20, 4.53, 3.68, 1.34. ¹³C NMR (300 MHz, Bruker, CDCl₃); δ ppm 164.61, 158.62, 157.65, 155.45, 133.48, 133.11, 131.26, 131.18, 131.02, 130.37, 130.15, 129.82, 118.98, 115.91, 114.21, 113.42, 96.20, 67.88, 65.99, 46.09, 12.60.

Hydrosilylation of Allyl-Rhodamine B. Allyl rhodamine B (50 mg), triethoxysilane (25 mg), and a catalytic amount of Pt/C were added to methanol (10 ml). The mixture was stirred at reflux temperature for one day, and then the Pt/C was removed via filtration through celite. The solvent of the filtrate was removed in vacuo to obtain 60.8 mg of hydrosilylated allyl-rhodamine B as a very sticky liquid (yield: 98.7%).

Preparation of Nanoparticles. Hydrosilylated allyl-rhodamine B (30 mg) and tetraethoxysilane (1.1 g) were added to a mixture of 28% NH₄OH (1 ml), deionized water (1 ml) and ethanol (50 ml). The mixture was stirred at room temperature for 3 hours. The reaction mixture was then subjected to centrifugation to give 0.1 g of silica-based fluorescent nanoparticles. The obtained nanoparticles have an average diameter of 30 to 50 nm when measured by TEM (tunneling electron microscopy), and have an average diameter of 30 to 50 nm when measured by SEM (scanning electron microscopy).

### Example 2

### Preparation of Fluorescein-Derived Silica-Based Fluorescent Nanoparticles

Silica-based nanoparticles are prepared in the same manner as in Example 1, except that 0.5 g of fluorescein was used instead of rhodamine B.

¹H NMR (300 MHz, Bruker, CDCl₃); δ ppm 8.25, 7.70, 7.33, 6.91, 6.78, 6.02, 5.60, 5.42, 5.11, 4.65, 4.46. ¹³C NMR (300 MHz, Bruker, CDCl₃); δ ppm 185.65, 165.00, 162.98, 158.90, 154.17, 149.98, 134.38, 132.69, 131.21, 131.90, 131.21, 130.95, 130.55, 130.50, 130.21, 129.94, 129.69, 128.91, 119.15, 118.70, 117.72, 114.97, 113.74, 105.74, 101.18, 69.44, 66.01.

### Example 3

### Preparation of Colorant Composition Including Silica-Based Fluorescent Nanoparticles

The nanoparticles of Example 1 having an average diameter of 50 nm were dispersed in 10 ml of ethanol or water. The obtained nanoparticles dispersed in the solvent exhibit a maximum absorption wavelength at about 560 nm and an emission wavelength at about 580 nm.

An aqueous solution of the silica-based fluorescent nanoparticles of Examples 1 and 2 were applied to a filtration paper (90 mm, ADVANTEC) to make a character, and then allowed to dry in air. Upon ultraviolet radiation, the character made by the solution of the nanoparticles of Example 1 exhibits red fluorescence, and the character made by the solution of the nanoparticles of Example 2 exhibits green fluorescence.

### Example 4

### Biochemical Marker Using the Silica-Based Fluorescent Nanoparticles

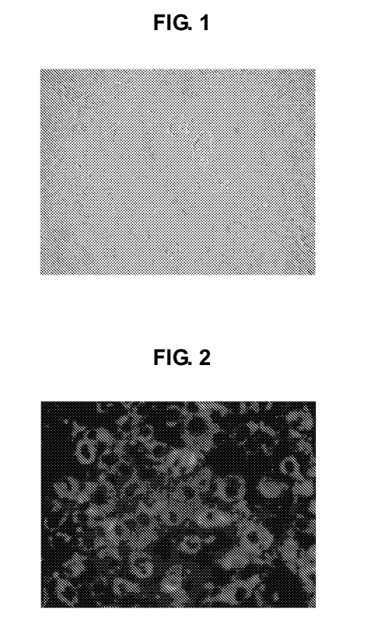

The nanoparticles of Example 1 having an average diameter of 50 nm were dispersed in a culture medium. The nanoparticles were incorporated into predetermined MC3T3-E1 cells in a concentration of 100 μg/ml. After a culture for 14 hours at 37° C. (5% CO₂), the cells were observed with an optical microscope and a fluorescence microscope. As shown in FIG. 1, when observed with an optical microscope, the existence of the fluorescent nanoparticles cannot be identified. As shown in FIG. 2, when observed with a fluorescence microscope, the existence of the fluorescent nanoparticles can be confirmed throughout all parts of the cells except the nuclei.

### Equivalents

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A composition comprising:
the reaction product of:
an organic silane of Formula SiR¹ₘX¹₄₋ₘ;
a fluorescent dye-silane compound of Formula D-L¹-(CH₂)ₙ—SiX²₃;
water; and
a hydrolysis catalyst;
wherein:
R¹ is a C₁-C₆ alkyl that is unsubstituted or substituted with one or more halogens or hydroxyl group, C₂-C₆ alkenyl that is unsubstituted or substituted with one or more halogens or OH groups, or an aryl group that is unsubstituted or substituted with one or more halogens or OH groups;
m is 0 or 1;
n is 3, 4, 5, 6, 7, 8, 9, 10, 11, or 12,
D is a radical having a fluorophore;
L1 is a bond, O, S, C(O)O, C(O)NR², SO₂O, C(O)S, C(S), or S₂;
R² is hydrogen, or a C₁-C₁₂ alkyl that is unsubstituted or is substituted with OH;
each X¹ and X² are independently a hydrolyzable substituent; and
the reaction product is a silica-based fluorescent nanoparticle comprising an outer surface comprising functional groups.

2. The composition of claim 1, wherein 80% or more of the functional groups on the outer surface of the silica-based fluorescent nanoparticle are OH groups.

3. The composition of claim 1, wherein D is a radical having a fluorophore derived from fluorescent dyes based on xanthene, benzo[a]xanthene, benzo[b]xanthene, benzo[c]xanthene, coumarin, benzocoumarin, alizarin, azo, phenoxazine, benzo[a]phenoxazine, benzo[b]phenoxazine, benzo[c]phenoxazine, naphthalimide, naphtholactam, azlactone, methyne, oxazine, thiazine, diketopyrrolopyrrole, quinacridone, thioepindoline, lactamimide, diphenylmaleimide, acetoacetamide, imidazothiazine, benzanthrone, phthalimide, benzotriazole, pyrimidine, pyrazine, or triazine.

4. The composition of claim 1, wherein a surface of the silica-based fluorescent nanoparticle comprises, a group that is —OR³ or —(CH₂)ₚY bonded to a silicon atom;
wherein:
Y is NR⁴R⁵R⁶, PO₂(OR⁷), or —(OCH₂CH₂)₄₋₁₅OR⁸,
R³, R⁴, R⁵, R⁶, R⁷, and R⁸ are each independently C₁-C₈ alkyl; and
p is an integer from 1 to 15.

5. The composition of claim 1, wherein 10% or less of the functional groups on the outer surface of the silica-based fluorescent nanoparticle are NH₂ groups.

6. The composition of claim 1, wherein R¹ is methyl, ethyl, or phenyl.

7. The composition of claim 1, wherein X¹ and X² are each independently halogen, C₁-C₆ alkoxy, or C₁-C₆ acyloxy.

8. The composition of claim 1, wherein D is a radical represented by Formula I, Formula II, Formula III, or Formula IV: 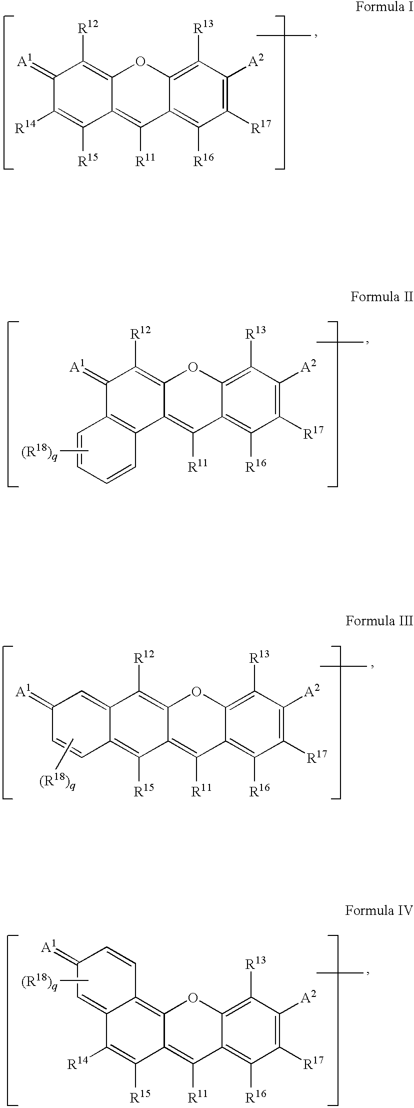
wherein:
A¹ is O, N-Z¹, or NZ¹Z²;
Z¹ and Z² are each independently H or C₁-C₈ alkyl, or Z¹ and R¹² join together to form a 5-, 6-, or 7-membered ring together with the atoms to which they are bonded, or Z¹ and R¹⁴ join together to form a 5-, 6-, or 7-membered ring together with the atoms to which they are bonded, or Z² and R¹⁴ join together to form a 5-, 6-, or 7-membered ring together with the atoms to which they are bonded;
A² is OZ³ or NZ⁴Z⁵;
Z³ is H, C₁-C₈ alkyl, or carboxy C₁-C₈ alkyl;
Z⁴ and Z⁵ are each independently H or C₁-C₈ alkyl, or Z⁴ and R¹³ join together to form a 5-, 6-, or 7-membered ring together with the atoms to which they are bonded, or Z⁴ and R¹⁷ join together to form a 5-, 6-, or 7-membered ring together with the atoms to which they are bonded, or Z⁵ and R¹⁷ join together to form a 5-, 6-, or 7-membered ring together with the atoms to which they are bonded;
q is an integer of 1 to 4,
R¹¹ is F, Cl, Br, I, CN, CF₃, C₁-C₈ alkyl, C₂-C₈ alkenyl, C₂-C₈ alkynyl, phenyl, naphthyl, or a group of Formula 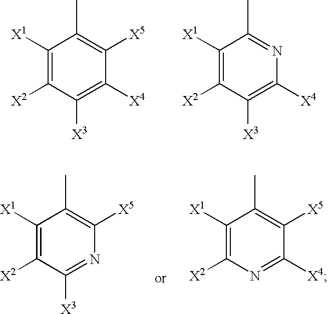
X¹, X², X³, X⁴, and X⁵ are independently H, F, Cl, Br, I, CN, CF₃, C₁-C₈ alkyl, C₁-C₈ alkoxy, C₁-C₈ alkylthio, C₂-C₈ alkenyl, C₂-C₈ alkynyl, C₁-C₈ alkylamido, SO₃H, sulfonate, or CO₂H, or X¹ and X², X² and X³, X³ and X⁴, or X⁴ and X⁵ join together form a phenyl group, together with the atoms to which they are bonded, which is unsubstituted or substituted with 1 to 4 F, Cl, Br, I, CN, CO₂H, SO₃H, OH, NH₂, with unsubstituted or substituted mono- or di(C₁-C₈ alkyl)amino, unsubstituted or substituted C₁-C₈ alkyl, unsubstituted or substituted C₁-C₈ alkylthio, or unsubstituted or substituted C₁-C₈ alkoxy;
R¹², R¹³, R¹⁴, R¹⁵, R¹⁶, R¹⁷, and R¹⁸ are independently H, F, Cl, Br, I, CN, CF₃, unsubstituted or substituted C₁-C₈ alkyl, unsubstituted or substituted C₁-C₈ alkylthio, unsubstituted or substituted C₁-C₈ alkoxy, phenyl, naphthyl, or heteroaryl, or R¹⁴ and R¹⁵, or R¹⁶ and R¹⁷ join to form a benzo group.

9. The composition of claim 1, wherein D is a coumarin radical represented by Formula V: 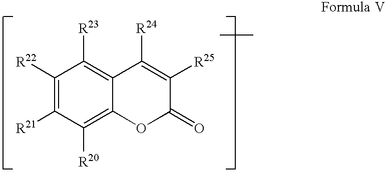
wherein:
R²⁰, R²¹, R²², R²³, R²⁴, and R²⁵ are independently H, C₁-C₈ alkyl, or NZ⁶Z⁷,
Z⁶ and Z⁷ are each independently H or C₁-C₈ alkyl, or R²⁰ and Z⁶; R²⁰ and Z⁷; R²² and Z⁶; or R²² and Z⁷ join together to form a 5-, 6-, or 7-membered ring together with atoms to which they are bonded, that may be unsubstituted or substituted.

10. The composition of claim 1, wherein ratio of the organic silane to the fluorescent dye-silane compound ranges from 1:1 to 100:1.

11. A method of preparing a silica-based florescent nanoparticle, comprising:
reacting a fluorescent dye of Formula D-A with a compound of Formula B—(CH₂)_{q′}—CH═CH₂ to produce a fluorescent dye derivative of Formula D-L1-(CH₂)_{q′}—CH═CH₂;
reacting the fluorescent dye derivative of Formula D-L1-(CH₂)_{q′}—CH═CH₂ with a silane compound of Formula HSiX²₃ to produce a fluorescent dye-silane compound of Formula D-L¹-(CH₂)ₙ—SiX²₃: and
polymerizing an organic silane of Formula SiR¹_{m′}X¹₄₋ₘ and the fluorescent dye-silane compound of Formula D-L¹-(CH₂)ₙ—SiX²₃ in the presence of water and a hydrolysis catalyst:
wherein
R¹ is a C₁-C₆ alkyl that is unsubstituted or substituted with one or more halogens or hydroxyl group, C₂-C₆ alkenyl that is unsubstituted or substituted with one or more halogens or hydroxyl group, or an aryl group that is unsubstituted or substituted with one or more halogens or hydroxyl group;
D is a radical having a fluorophore;
each X¹ and X² are independently a hydrolyzable substituent;
L1 is a bond, O, S, C(O)O, C(O)NR², SO₂O, C(O)S, C(S), or S₂;
A is COOH, OH, SO₃H, CO—CH₂-halogen, CH═CH₂ or SH;
B is OH, NHR², F, Cl, Br, I, or SH;
q′ is an integer of 1 to 10;
n is an integer of 3 to 12;
m′ is 0 or 1; and
R² is hydrogen, C₁-C₁₂ alkyl, or hydroxy-substituted C₁-C₁₂ alkyl;
with the proviso that A and B are selected in such a manner as to be able to react with each other.

12. The method of claim 11, wherein L¹ is C(O)O, A is C(O)OH, and B is F, Cl, Br, I, or OH.

13. The method of claim 11, wherein the fluorescent dye derivative of Formula D-L1-(CH₂)_{q′}—CH═CH₂ is reacted with the silane compound of Formula HSiX²₃ in a ratio of from 1:0.5 to 1:5.

14. A compound represented by Formula D-L1-(CH₂)q′-CH═CH₂ wherein:
D is a radical having a fluorophore;
L1 is a bond, O, S, C(O)O, C(O)NR², SO₂O, C(O)S, C(S), or S₂; and
q′ is an integer of 1 to 10.

15. The compound of claim 14, wherein D is a radical represented by Formula I, Formula II, Formula III, or Formula IV: 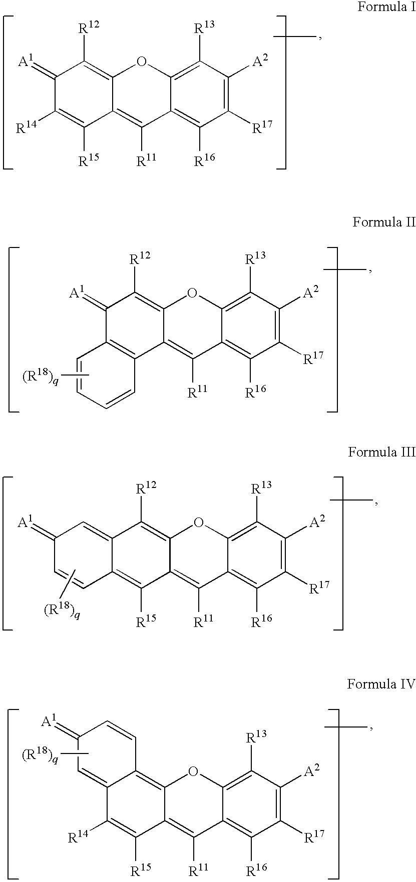
wherein:
A¹ is O, N-Z¹, or NZ¹Z²;
Z¹ and Z² are each independently H or C₁-C₈ alkyl, or Z¹ and R¹² join together to form a 5-, 6-, or 7-membered ring together with the atoms to which they are bonded, or Z¹ and R¹⁴ join together to form a 5-, 6-, or 7-membered ring together with the atoms to which they are bonded, or Z² and R¹⁴ join together to form a 5-, 6-, or 7-membered ring together with the atoms to which they are bonded;
A² is OZ³ or NZ⁴Z⁵;
Z³ is H, C₁-C₈ alkyl, or carboxy C₁-C₈ alkyl;
Z⁴ and Z⁵ are each independently H or C₁-C₈ alkyl, or Z⁴ and R¹³ join together to form a 5-, 6-, or 7-membered ring together with the atoms to which they are bonded, or Z⁴ and R¹⁷ join together to form a 5-, 6-, or 7-membered ring together with the atoms to which they are bonded, or Z⁵ and R¹⁷ join together to form a 5-, 6-, or 7-membered ring together with the atoms to which they are bonded;
q is an integer of 1 to 4;
R¹¹ is F, Cl, Br, I, CN, CF₃, C₁-C₈ alkyl, C₂-C₈ alkenyl, C₂-C₈ alkynyl, phenyl, naphthyl, or a group of Formula 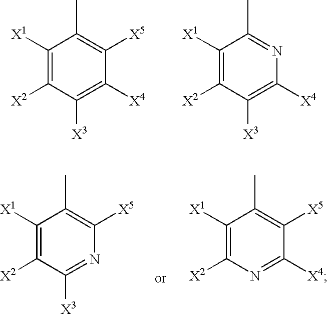
X¹, X², X³, X⁴, and X⁵ are independently H, F, Cl, Br, I, CN, CF₃, C₁-C₈ alkyl, C₁-C₈ alkoxy, C₁-C₈ alkylthio, C₂-C₈ alkenyl, C₂-C₈ alkynyl, C₁-C₈ alkylamido, SO₃H, sulfonate, or CO₂H, or X¹ and X², X² and X³, X³ and X⁴, or X⁴ and X⁵ join together form a phenyl group, together with the atoms to which they are bonded, which is unsubstituted or substituted with 1 to 4 F, Cl, Br, I, CN, CO₂H, SO₃H, OH, NH₂, unsubstituted or substituted mono- or di(C₁-C₈ alkyl)amino, unsubstituted or substituted C₁-C₈ alkyl, unsubstituted or substituted C₁-C₈ alkylthio, or unsubstituted or substituted C₁-C₈ alkoxy;
R¹², R¹³, R¹⁴, R¹⁵, R¹⁶, R¹⁷, and R¹⁸ are independently H, F, Cl, Br, I, CN, CF₃, unsubstituted or substituted C₁-C₈ alkyl, unsubstituted or substituted C₁-C₈ alkylthio, unsubstituted or substituted C₁-C₈ alkoxy, phenyl, naphthyl, or heteroaryl, or R¹⁴ and R¹⁵, or R¹⁶ and R¹⁷ join to form a benzo group.

16. The compound of claim 14, wherein, wherein D is a radical represented by Formula V: 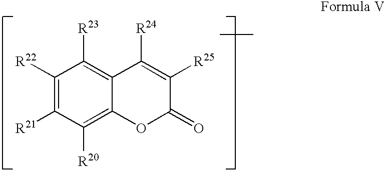
wherein R²⁰, R²¹, R²², R²³, R²⁴, and R²⁵ are independently H, C₁-C₈ alkyl, or NZ⁶Z⁷,
Z⁶ and Z⁷ are each independently H or C₁-C₈ alkyl, or R²⁰ and Z⁶; R²⁰ and Z⁷; R²² and Z⁶; or R²² and Z⁷ join together to form a 5-, 6-, or 7-membered ring together with atoms to which they are bonded, that may be unsubstituted or substituted.

17. A colorant composition comprising the composition of claim 1.

18. The colorant composition of claim 17, which includes a fluorescent ink, a fluorescent paint or a fluorescent paste.

19. The colorant composition of claim 17, which is used as a biochemical marker.

20. A method of coloring an article by using the composition of claim 1.

21. A method of biochemically staining a cell by using the composition of claim 1.
